(19) United States
(12) Patent Application Publication
(10) Pub. No.: US 2018/0279978 A1
(43) Pub. Date: Oct. 4, 2018

(54) MOLECULAR BREAST IMAGING SYSTEM WITH GANTRY ASSEMBLY FOR LATERAL-FACING ULTRASOUND

(71) Applicant: MAYO FOUNDATION FOR MEDICAL EDUCATION AND RESEARCH, ROCHESTER, MN (US)

(72) Inventor: MICHAEL K O'CONNOR, ROCHESTER, MN (US)

(21) Appl. No.: 15/938,231

(22) Filed: Mar. 28, 2018

Related U.S. Application Data
(60) Provisional application No. 62/478,273, filed on Mar. 29, 2017.

(51) Int. Cl. A61B 6/00; A61B 6/04; A61B 8/08; A61B 8/00; A61B 10/00; A61B 17/34

(52) U.S. Cl. CPC A61B 6/4417; A61B 6/0414; A61B 6/4057; A61B 6/4258; A61B 6/502; A61B 6/5247; A61B 2010/045; A61B 8/403; A61B 8/4416; A61B 8/5261; A61B 10/0041; A61B 17/3403; A61B 8/0825

(57) ABSTRACT

A combined molecular breast imaging (“MBI”) and ultrasound system is described. A gantry assembly provides for introduction of a lateral facing ultrasound probe while a subject's breast is maintained under compression by the MBI system.

### CROSS-REFERENCE TO RELATED APPLICATIONS

This application claims the benefit of U.S. Provisional Patent Application Ser. No. 62/478,273, filed on Mar. 29, 2017, and entitled “MOLECULAR BREAST IMAGING SYSTEM WITH GANTRY ASSEMBLY FOR LATERAL-FACING ULTRASOUND,” which is herein incorporated by reference in its entirety.

### BACKGROUND

Breast cancer screening has been recommended for many decades, particularly in women over the age of fifty. The combination of early detection and improved therapy in the U.S. has resulted in a significant reduction in breast cancer mortality, with similar reductions being observed in other countries. Despite the success of screening mammography, however, it is also recognized that mammography is a less than perfect screening method. The limitations of mammography are particularly evident in women with mammographically dense breasts. It has been shown that the sensitivity of mammography decreases with increasing mammographic density, and is less than fifty percent for women with an extremely dense breast pattern on a mammogram.

The reduced sensitivity of mammography with increasing mammographic density is compounded by the fact that increased density is a significant risk factor for breast cancer. Given that a dense breast pattern occurs more frequently in younger women, this factor significantly diminishes the value of mammography in the screening of young women who have a high familial risk of breast cancer.

A second major limitation to screening mammography is in the evaluation of women at high risk of breast cancer. Numerous studies have demonstrated that in women with a high genetic risk of breast cancer, mammography has a sensitivity of between 33-43 percent. Most of these studies have been performed in women with an average age of forty, so part of the explanation for the poor performance of mammography in these studies may be due to the presence of dense breast patterns in a significant percentage of the mammographic images.

A possible solution to the problem of the detection of breast lesions in dense breast tissue is to use ultrasound in such patients. Ultrasound is attractive for supplemental screening because it is widely available, is well-tolerated by patients, and involves no radiation. However, while supplemental ultrasound screening uncovers more breast cancers, it also substantially increases the risk of a false positive cancer finding and unnecessary biopsy. Hence, the use of whole-breast ultrasound as a sole identifier of breast malignancies is questionable. Even in combination with mammography, the two anatomical techniques have significant limitations. It would be of considerable benefit to provide a complementary method that provides functional information about lesions seen on ultrasound. Such a method would significantly reduce the number of false positive cases, and allow the radiologist to evaluate those lesions that demonstrate both a functional and anatomical abnormality.

Over the last several years, a number of nuclear medicine-based technologies have been developed that have application in breast imaging. Included in these are positron emission mammography (“PEM”) and molecular breast imaging (“MBI”). In PEM the breast is compressed between two opposing detectors and the 511 keV gamma rays emitted by a positron emitting radiopharmaceutical, such as F-18 fluoro-deoxyglucose, are detected by coincidence imaging between the two opposing detectors. The PEM images provide an image of glucose utilization by breast tissue and have been shown to be capable of detecting small cancers in the breast. Unlike anatomical techniques such as mammography and ultrasound, PEM is not influenced by dense breast tissue.

The second nuclear medicine-based technique is MBI. This technology employs one or two small gamma cameras. The breast is compressed between a camera and a compression paddle, or between two gamma cameras, and radiation emitted by single-photon radiopharmaceutical(s) (for example, Tc-99m sestamibi) is detected after collimation. MBI is a planar imaging technique without tomographic capability; however, information from two opposing gamma cameras can be used to calculate the depth of a functional abnormality in the MBI images. The MBI system has been shown to have a very high sensitivity, for example in some cases greater than ninety percent, for the detection of lesions smaller than ten millimeters. In addition, it has been found that, in some cases, MBI can detect three times as many cancers as digital and analog mammography in asymptomatic women at increased risk of breast cancer.

Beyond sensitivity differences, technologies that provide functional images of the breast, such as MBI, can detect lesions not visible with conventional mammography. Likewise, certain benign breast conditions may result in a false positive finding on MBI, and this uptake can be readily identified as a benign process from the anatomical information available in ultrasound. Currently it is not practical to fuse anatomical images from ultrasound systems and functional images from MBI.

Ultrasound requires that the patient lie supine and a handheld scanner is then used to scan the breast. MBI is usually performed with the patient seated and the breast lightly compressed between the gamma cameras or camera and paddle. MBI employs light compression forces (for example 10-15 pounds of force) with imaging times in the 5-10 minute range. The imaging procedure is generally considered to be substantially pain-free. Because of the differences in patient orientation used in MBI and ultrasound imaging, the shape of the breast tissue is significantly different between the two modalities and, hence, correlation of an anatomical abnormality with a functional abnormality becomes difficult. Therefore, accurate co-registration of anatomical images from ultrasound and functional information from MBI is not currently possible.

Over the last few years, several entities have worked on the development of whole-breast ultrasound (“WUS”) systems. The main purpose of this development was to reduce the dependence of image quality on the technologist or radiologist, and provide a more reproducible imaging technique. The WUS systems are designed to image the patient in the supine position in a manner comparable to that of conventional breast ultrasound. If the patient is not supine, then the WUS system suffers from a loss in the achievable coverage of the breast tissue. Therefore, while WUS systems provide better coverage in non-supine patient positions than traditional ultrasound imaging, they are still limited in their applicability to combination with imaging modalities that require non-supine patient positions, such as MBI.

It would therefore be desirable to provide an MBI system that would allow the acquisition of both anatomical and functional images of the breast, such images being amenable to co-registration so that accurate and reliable assessments of the presence of cancerous lesions in the breast can be made. Additionally, it would be desirable to provide an MBI system that would also allow for breast biopsies to be performed under the guidance of ultrasound.

### SUMMARY OF THE DISCLOSURE

The present disclosure addresses the aforementioned drawbacks by providing a molecular breast imaging (“MBI”) system that includes a support structure, a first gamma ray detector, a second gamma ray detector, and a gantry assembly for aligning an ultrasound probe with a region-of-interest relative to the first and second gamma ray detectors. The first gamma ray detector is operably coupled to the support structure and extends to define a first imaging plane, and the second gamma ray detector is operably coupled to the support structure and extends to define a second imaging plane. The gantry assembly is coupled to the support structure and positioned between the first gamma ray detector and the second gamma ray detector. The gantry assembly includes a semicircular gantry, a vertical support member, and a support arm. The semicircular gantry extends in a gantry plane parallel to one of the first imaging plane or the second imaging plane. The vertical support member is coupled to the semicircular gantry and extends in a direction perpendicular to the gantry plane. The vertical support member is translatable along an extent of the semicircular gantry. The support arm operatively engages the vertical support member and extends in a lateral direction that is parallel to one of the first imaging plane or the second imaging plane. The support arm is operable to adjust a height of the support arm relative to the semicircular gantry and to adjust a lateral position of the support arm relative to the vertical support member. The support arm is also configured to receive an ultrasound probe at its distal end.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 illustrates an example molecular breast imaging (“MBI”) system.

FIG. 2 illustrates two radiation detectors (e.g., gamma ray detectors) used in an MBI system and their respective imaging planes.

FIG. 3 illustrates an example of a gantry assembly for providing a lateral-facing ultrasound probe to an MBI system.

FIG. 4 is a side view of an example gantry assembly according to FIG. 3 coupled to an MBI system.

FIG. 5 is a plan view of an example gantry assembly according to FIG. 3 coupled to an MBI system.

### DETAILED DESCRIPTION

Described here are combined molecular breast imaging (“MBI”) and ultrasound systems. The systems described in the present disclosure allow for accurate coregistration of information obtained from MBI with that from ultrasound, which provides for ultrasound-guided biopsies.

Current schemes for radio-guided breast biopsy are limited in their ability to incorporate additional information from x-ray or ultrasound imaging. Thus, the schemes developed for positron emission mammography (“PEM”) and breast specific gamma imaging (“BSGI”) only provide a conventional imaging scheme to localize a lesion, and neither of these systems incorporates any innovative features to enhance or accelerate the biopsy process. With typical imaging times for both PEM and BSGI at around ten minutes per view, the biopsy process can become a lengthy procedure depending on how many images need to be acquired to confirm correct placement of the biopsy needle.

The MBI system described in the present disclosure provides anatomical correlation information, which is helpful for aiding a clinician in the decision making process. In breast imaging, the variability in breast position between different modalities can make it difficult to correlate findings across these modalities, particularly in complex cases where both benign and malignant tissue regions may be present. It is contemplated that the ability to co-register an area of increased uptake on an image obtained with MBI with the corresponding area on an ultrasound image will enable physicians to better determine the nature of the lesion and the appropriate course of action.

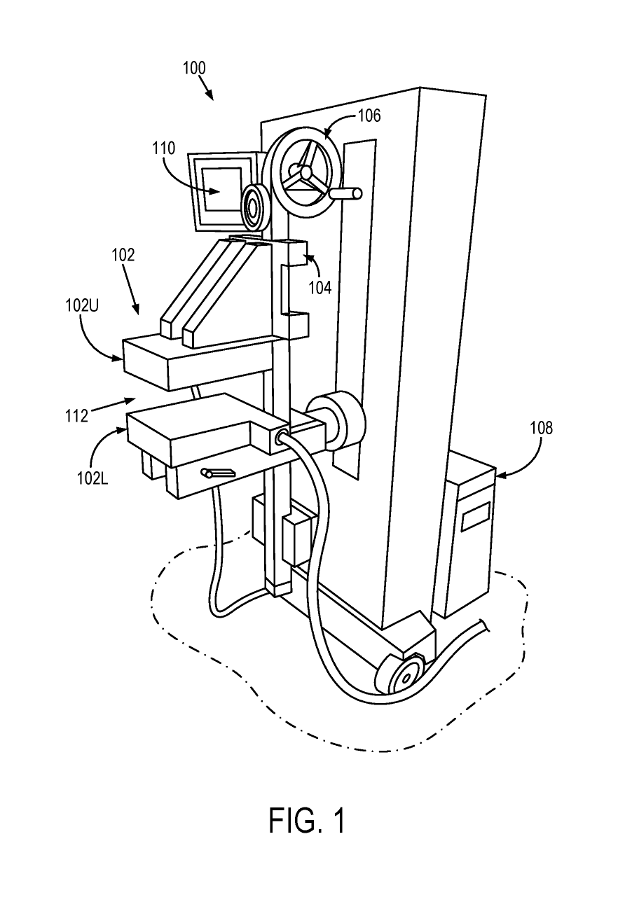

Referring to FIG. 1, a non-limiting example of an MBI system 100 includes two opposing radiation detectors 102. The radiation detectors may be cadmium zinc telluride (“CZT”) detectors, or may be other suitable radiation detectors, such as other semiconductor radiation detectors. The detectors 102 include an upper detector 102U and a lower detector 102L. Examples of MBI systems and methods for their use are described, for example, in U.S. Pat. No. 8,923,952, entitled “System and Method for Quantitative Molecular Breast Imaging,” which is herein incorporated by reference in its entirety. As one non-limiting example, each detector 102U, 102L may be 20-24 centimeters (“cm”) by 16-20 cm in size and mounted on a modified upright type mammographic gantry 104. In one configuration, the detectors 102 are Lumagem® 3200S high-performance, solid-state cameras from Gamma Medica-Ideas, Inc., having a pixel size of 1.6 millimeters (“mm”) (Lumagem® is a trademark of Gamma Medica-Ideas, Inc., Northridge, Calif.).

The relative position of the detectors 102 can be adjusted using a user control 106. The detectors 102 are designed to serve as a compression mechanism. Accordingly, this system configuration reduces the maximum distance between any lesion in the breast and either detector 102 to one-half of the total breast thickness, potentially increasing detection of small lesions without additional imaging time or dose. The MBI system 100 includes data-processing circuitry (such as a processor) 108 for processing the signals acquired by the detectors 102 to produce an image, which may be displayed on an associated display 110.

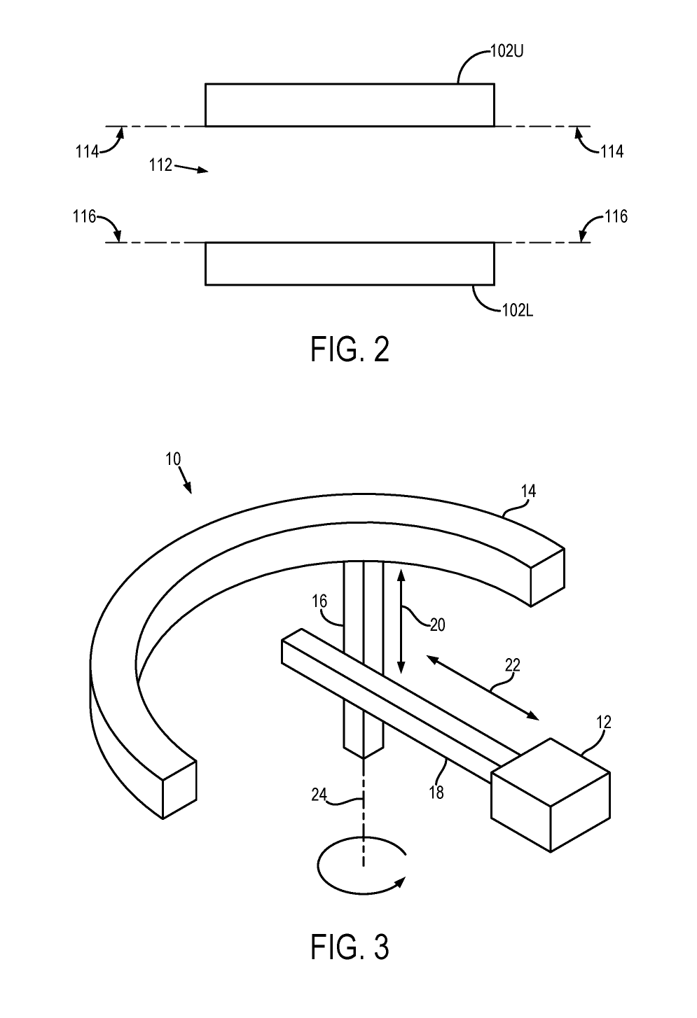

In general, the detectors 102 are arranged so as to form an examination region 112 there between. The examination region 112 is defined with respect to a first imaging plane 114 and a second imaging plane 116, as shown in FIG. 2. The first imaging plane 114 is defined, for example, by the extension of the upper detector 102U along the examination region 112, and the second imaging plane 116 is defined, for example, by the extension of the lower detector 102L along the examination region 112.

The systems described in the present disclosure implement a lateral-facing ultrasound probe that is coupled to a gantry positioned between the upper and lower detectors of an MBI system. This configuration allows for ultrasound imaging of a subject's breast without needing to move the MBI detectors.

As shown in FIG. 3, an example of a gantry assembly 10 supports an ultrasound probe 12 and provides for aligning the ultrasound probe 12 with a region-of-interest, such as a region-of-interest identified in an MBI image. The gantry assembly 10 includes a semicircular gantry 14 that extends in a plane coplanar to the detectors 102. For example, the semicircular gantry 14 can extend in a plane that is coplanar to one or both of the first imaging plane 114 and second imaging plane 116.

A vertical support member 16 is coupled to the semicircular gantry 14 and extends in a direction perpendicular to the plane in which the semicircular gantry 14 extends. For example, the vertical support member 16 can extend in a direction perpendicular to one or both of the first imaging plane 114 and the second imaging plane 116. The vertical support member 16 is operable to be moved along the semicircular gantry 14 through a number of different angular positions so as to allow adjustment of the line-of-sight of the ultrasound probe 12 about the subject's breast without needing to move the MBI system detectors. As one example, the semicircular gantry 14 can be sized to provide tracking of the vertical support member 16 through a 180 degree arc around a subject's breast positioned in the MBI system 100.

A support arm 18 engages the vertical support member 16 and extends in a lateral direction, such as a direction that is orthogonal to the vertical support member 16. The support arm 18 is operable to translate along the vertical support member 16 (e.g., the support arm 18 can be raised or lowered along the vertical support member 16) such that the height of the support arm 18 relative to the semicircular gantry 14 can be adjusted, as indicated by arrows 20. This height adjustment allows for the ultrasound probe 12 to be aligned with the estimated depth of the region-of-interest in the subject's breast. The support arm 18 is also operable to translate along the lateral direction (i.e., the direction along which the support arm extends), as indicated by arrows 22. This latter adjustment allows the ultrasound probe 12 to be retracted away from the subject's breast during an examination without needing to move the MBI system detectors. In some configurations, the vertical support member 16 can also be made to rotate about its own longitudinal axis 24 so as to provide for rotation of the support arm 18 about that longitudinal axis 24.

The ultrasound probe 12 is coupled to the distal end of the support arm 18. In use, the gantry assembly 10 can be adjusted to provide contact between the ultrasound probe 12 and a surface of the subject's breast. The multiple degrees of freedom provided by the gantry assembly 10 allow for the ultrasound probe 12 to be moved into alignment with a region-of-interest in the subject's breast without needing to move the MBI system detectors or the subject's breast.

The ultrasound probe 12 can include any suitable ultrasound transducer. In some implementations, the ultrasound probe operates with a transducer frequency that is sufficiently low so as to allow for depth penetration of up to 10 cm in tissue. In some other implementations, the ultrasound probe is operated at a higher transducer frequency, and other techniques can be implemented to retain adequate spatial resolution at depths of up to 10 cm in tissue.

Because the support arm 18 is coupled to the semicircular gantry 14, which acts as a fixed point of reference, the position of the ultrasound probe 12 is known. Likewise, the distance of the ultrasound probe 12 along the radius of the semicircular gantry 14 allows for the calculation of the location of the transducer face of the ultrasound probe 12. Vertical adjustment of the ultrasound probe 12 via adjustment of the support arm 18 along the length of the vertical support member 16 allows for the imaging plane of the ultrasound probe 12 to be aligned with the estimated tumor depth. These three variable can be used to coregister the lesion, or region-of-interest, seen on an MBI image to an exact location on the ultrasound images.

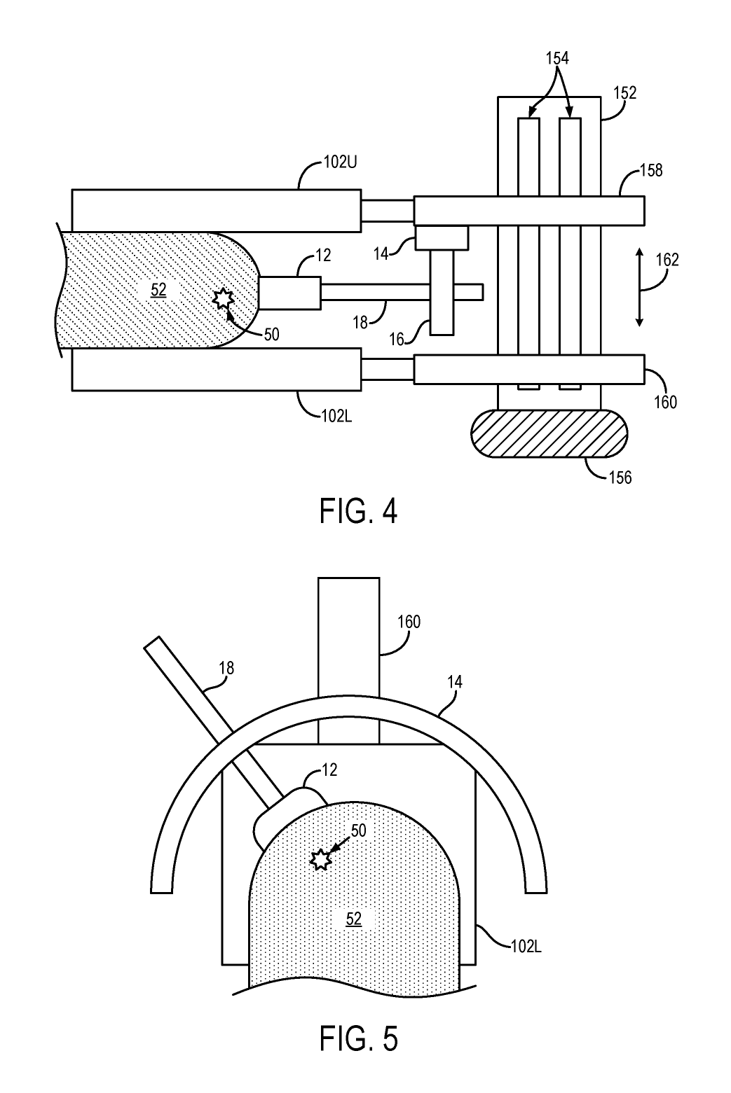

FIGS. 4 and 5 illustrate a side view and a top view, respectively, of an example MBI system 100 in which a gantry assembly 10 is being used to align an ultrasound probe 12 with a region-of-interest 50 in a subject's breast 52. The MBI system 100 includes a support structure 152 having formed therein one or more tracks 154. The support structure 152 is coupled to a gantry 156 that allows for the MBI system 100 to be positioned about the patient in a number of different orientations. Coupled to the tracks 154 are an upper support arm 158 and a lower support arm 160. The upper detector 102U is coupled to the upper support arm 158, and the lower detector head 102L is coupled to the lower support arm 160. A motor drives the upper support arm 158 so that the upper detector 102U is moved along a direction 162 towards or away from the lower detector 102L to provide compression of the subject's breast 52.

The gantry assembly 10 can be coupled to the upper support arm 158 of the MBI system 100, or in an alternative configuration can be coupled to the lower support arm 160 of the MBI system 100. The vertical support member 16 is generally sized such that its length is no longer than the spacing between the upper support arm 158 and the lower support arm 160 when the maximum expected amount of compression is being provided. In this manner, the movement of the vertical support arm 16 along the extent of the semicircular gantry 14 (e.g., along the circumferential extent of the semicircular gantry 14) will not impinge upon either the upper support arm 158 or the lower support arm 160.

Functional imaging of the breast can be performed using the aforementioned MBI system, which permits a calculation of an in-plane location of a lesion or other region-of-interest in the breast, as well as its depth and relative uptake of an administered radionuclide. Following completion of the MBI acquisition, the ultrasound probe 12 may be moved into position by adjusting the gantry assembly 10. While moving the ultrasound probe 12 into position (e.g., into alignment with the region-of-interest in the subject's breast), the detectors 102 of the MBI system 100 remain in physical contact with the subject's breast. This process allows the maintenance of constant compression of the breast between the detectors 102 while the MBI system 100 converts from a molecular imaging mode to a combined MBI/ultrasound imaging mode. Moreover, this constant compression of the breast mitigates subject movement while switching between operational modes.

After the ultrasound probe 12 has been moved into position, the MBI acquisition can be repeated and ultrasound probe 12 operated to obtain high resolution images of the region-of-interest identified on the MBI images. Upon completion of both the molecular and ultrasound imaging acquisitions, the MBI and ultrasound images can be co-registered. As mentioned above, the images can be co-registered based on knowledge of the angle, height, and depth of the ultrasound probe 12.

Using the MBI system configuration described in the present disclosure, functional and anatomical information can be obtained sequentially or simultaneously from the two imaging modalities.

The present disclosure has described one or more preferred embodiments, and it should be appreciated that many equivalents, alternatives, variations, and modifications, aside from those expressly stated, are possible and within the scope of the invention.

1. A molecular breast imaging (MBI) system comprising:
a support structure;
a first gamma ray detector operably coupled to the support structure and extending to define a first imaging plane;
a second gamma ray detector operably coupled to the support structure and extending to define a second imaging plane;
a gantry assembly for aligning an ultrasound probe with a region-of-interest relative to the first and second gamma ray detectors, the gantry assembly being coupled to the support structure and positioned between the first gamma ray detector and the second gamma ray detector, the gantry assembly comprising:
a semicircular gantry that extends in a gantry plane parallel to one of the first imaging plane or the second imaging plane;
a vertical support member coupled to the semicircular gantry and extending in a direction perpendicular to the gantry plane, the vertical support member being translatable along an extent of the semicircular gantry;
a support arm operatively engaging the vertical support member and extending in a lateral direction that is parallel to one of the first imaging plane or the second imaging plane, wherein the support arm is operable to adjust a height of the support arm relative to the semicircular gantry and to adjust a lateral position of the support arm relative to the vertical support member; and
wherein the support arm is configured to receive an ultrasound probe at its distal end.

2. The MBI system as recited in claim 1, further comprising an ultrasound probe coupled to the distal end of the support arm.

3. The MBI system as recited in claim 2, wherein the ultrasound probe is coupled to the distal end of the support arm such that the ultrasound probe is oriented to transmit ultrasound along the lateral direction.

4. The MBI system as recited in claim 1, wherein the support arm is rotatable about a pivot axis such that the support arm can be rotated toward and away from the gantry plane.

5. The MBI system as recited in claim 1, wherein the vertical support member is rotatable about a longitudinal axis such that the support arm can be rotated about the longitudinal axis through angular positions in the gantry plane.

6. The MBI system as recited in claim 1, wherein the first gamma ray detector is coupled to the support structure via a first support arm and the second gamma ray detector is coupled to the support structure via a second support arm, and wherein the gantry assembly is coupled to one of the first support arm or the second support arm.

7. The MBI system as recited in claim 6, wherein at least one of the first support arm and the second support arm is operable to translate along the support structure to alter a spacing between the first gamma ray detector and the second gamma ray detector.

8. The MBI system as recited in claim 7, wherein the vertical support member has a length shorter than a minimum spacing between the first gamma ray detector and the second gamma ray detector.

9. A gantry assembly for aligning an ultrasound probe with a region-of-interest relative to gamma ray detectors in a molecular breast imaging (MBI) system, comprising:
a semicircular gantry that extends in a gantry plane;
a vertical support member coupled to the semicircular gantry and extending in a direction perpendicular to the gantry plane, the vertical support member being translatable along an extent of the semicircular gantry;
a support arm operatively engaging the vertical support member and extending in a lateral direction that is parallel to the gantry plane, wherein the support arm is operable to adjust a height of the support arm relative to the semicircular gantry and to adjust a lateral position of the support arm relative to the vertical support member.

10. The gantry assembly as recited in claim 9, wherein the support arm is rotatable about a pivot axis such that the support arm can be rotated toward and away from the gantry plane.

11. The gantry assembly as recited in claim 9, wherein the vertical support member is rotatable about a longitudinal axis such that the support arm can be rotated about the longitudinal axis through angular positions in the gantry plane.

12. The gantry assembly as recited in claim 9, further comprising an ultrasound probe coupled to a distal end of the support arm.

13. The gantry assembly as recited in claim 12, wherein the ultrasound probe is coupled to the distal end of the support arm such that the ultrasound probe is oriented to transmit ultrasound along the lateral direction.
